(19) United States
(12) Patent Application Publication
(10) Pub. No.: US 2019/0105474 A1
(43) Pub. Date: Apr. 11, 2019

(54) DRAINAGE CATHETER WITH BALLOON

(71) Applicant: Sheibley Medical LLC, Billings, MT (US)

(72) Inventor: Dan Sheibley, Billings, MT (US)

(21) Appl. No.: 15/727,614

(22) Filed: Oct. 8, 2017

(51) Int. Cl. A61M 27/00

(52) U.S. Cl. CPC A61M 27/00; A61M 25/007; A61M 2210/1089

(57) ABSTRACT

A catheter having an elongated tube with a central lumen, a proximal end, and a distal end terminating in a tip; a balloon and a balloon lumen that is in fluid communication with the balloon, the balloon being situated at a point along the elongated tube between the proximal and distal ends of the elongated tube; and a catheter hub that is connected to the proximal end of the elongated tube, the catheter hub being in fluid communication with the central lumen and the balloon lumen. The elongated tube has one or more side holes between the balloon and the tip of the distal end of the elongated tube or an end hole at the tip of the elongated tube. The elongated tube also has one or more side holes between the balloon and the hub. There is no fluid communication between the central lumen and the balloon lumen.

### BACKGROUND OF THE INVENTION

### 1. Field of the Invention

The present invention relates generally to the field of medical devices, and more particularly, to a balloon catheter for use in treating biliary duct stenosis and ureteral stenosis.

### 2. Description of the Related Art

The present invention is intended to provide a more effective treatment for biliary duct stenosis and ureteral stenosis than existing devices and methods. Treatment of these conditions typically requires multiple repeat visits approximately four weeks apart over a span of multiple months, with further inconvenience and discomfort to the patient as well as repeated costs of sedation, interventional radiologist procedural costs, interventional radiologic suite usage, nursing and ancillary staff and materials (drainage catheters, balloon dilation catheters, wires, sheaths and injected contrast). The treatment pathway for biliary duct stenosis is described below, but the procedure for treating ureteral strictures is comparable.

The liver produces bile, which is excreted into the bile ducts, a branching network of tubes that join together as they progress from the periphery of the liver toward the central portion of the liver known as the hilum. At the hilum these ducts join into a single conduit known as the common bile duct. By conventional anatomy, this common bile duct passes into the pancreatic head, joins with the pancreatic duct, and then joins with the duodenum (first segment of small bowel) and allows the passage of bile into the small bowel. If this common bile duct or any portion of duct within the liver becomes narrowed, it either ceases or greatly diminishes the amount of bile that can drain through the duct and into the bowel. The results of this occurrence are jaundice, itching, pain and even sepsis, as this stagnant collection of bile can become infected.

The bile ducts can become narrowed (referred to in medical terminology as a “stricture”) for a number of reasons, including chronic inflammation of the bile ducts (cholangitis) and the formation of scar tissue due to surgical procedures. Both fibrosis and scar tissue can close down the inside passageway of the bile duct. Treatment of this condition requires multiple repeat procedures over multiple months to gradually stretch and remodel the duct to a diameter that is closer to the patient's native condition.

The conventional procedure for treating bile duct stenosis requires accessing the biliary tree with a needle through the skin surface, after which a thin wire (called a “guide wire”) is navigated through the biliary tree from the small peripheral branch that was accessed with the needle to the level of the stricture. The wire is passed through the stricture and out the end of the duct into the bowel. A catheter with a balloon is mounted over this wire and advanced to the level of the stricture. The balloon is then dilated temporarily, thereby stretching and causing minor trauma to the strictured segment of bile duct. Next, a drainage catheter with multiple side holes is advanced over the wire so that there are drainage holes both above and below the level of the stricture. The positioning of these holes allows bile produced in the liver from above or upstream of the stricture to pass into the central lumen of the drainage catheter and out the side holes or end hole of the catheter beyond or downstream of the stricture. The patient is then discharged and left with the drain in place for approximately four weeks. The purpose of the catheter is not only to facilitate drainage of the liver but also to serve as scaffolding around which the newly dilated strictured bile duct can collapse and heal over the four to six weeks' time until to the subsequent procedure.

After approximately four weeks with the catheter in place, the patient returns to the hospital, and the procedure is performed again. Because access into the bile ducts has already been obtained, a wire is advanced into the indwelling drainage catheter, which is subsequently removed. A radiocontrast agent is injected into the patient's bile ducts under fluoroscopy to show the residual narrowing of the bile ducts. A balloon dilation catheter (usually with a balloon of larger diameter than the previously used balloon) is then advanced over the wire, the balloon is temporarily inflated, and the balloon catheter is removed. Next, a new drainage catheter of larger caliber than the one previously used is advanced over the wire across the stenosis. This new, larger diameter catheter serves as scaffolding around which the further dilated strictured bile duct can collapse and heal over yet another period of time.

This balloon dilation and drainage catheter upsizing procedure is repeated multiple times over four- to six-week periods until the bile duct is at a larger, near native, caliber and able to maintain patency without a catheter holding it open. Returning to the hospital for repeat procedures requires substantial time, risk and costs to the patient. These include drainage catheter, balloon catheter, wire and contrast costs, risks of and costs of medicine for provision of sedation during each procedure, substantial cost for the interventional radiologist's time (MD), nursing and ancillary staff costs, and costs for the technical use of the equipment in the interventional radiology suite.

It is this problem of complex and repeat visits with substantial repeat costs of time, discomfort (with increasing the size of the catheter) and money to the patient that the present invention is intended to solve. The present invention is a new hybrid drainage and balloon dilation catheter that would allow the patient to undergo the initial access and first dilation with placement of the hybrid catheter at the hospital and then to continue to drain at home while repeating the balloon dilation that had been performed during the initial procedure. Performance of this balloon dilation procedure at home, for however many times the treating physician prescribes, would allow the patient to maintain the benefits of the initial balloon dilation (performed at the hospital) and to slowly increase the degree of balloon dilation in an outpatient setting. This would be done simply by inflating the balloon mounted on the drainage catheter on a set schedule with a set volume of fluid.

This repeated, more frequent stretching (much more frequent than once every four weeks) would serve to accelerate the time until bile duct patency were achieved. Being able to do this in an outpatient setting also saves a significant amount of time and money. An additional benefit is that the original, smaller diameter drainage catheter could be used as the balloon dilates to the desired larger diameter, and it would not be necessary to rely upon a larger diameter catheter to maintain the tract. This saves the patient from additional discomfort incurred with the traditional treatment pathway.

Patients and home caregivers routinely manage tubes with balloons on them, such as gastrostomy tubes (feeding tubes) and Foley catheters (bladder drainage catheters). Manipulating balloons on those catheters involves attachment of a saline syringe to a second hub on the catheter connected to the balloon and injecting or withdrawing fluid. Additionally, people with indwelling venous access catheters (basically large semi-permanent intravenous catheters) are very commonly responsible for and able to manage flushing their catheters. To perform the functions necessary to repeatedly dilate the balloon on the hybrid drainage/dilation catheter would be an analogous and reasonable task for patients.

Some improvements to catheters have been patented over the years, but none of these inventions embodies the structural details of the present invention. For example, U.S. Pat. No. 4,222,384 (Birtwell, 1980) discloses an improve Foley-type urethral catheter in which the tip of the catheter is molded in one integral piece to include drainage eyes and a rearwardly extending balloon portion. U.S. Pat. No. 4,813,935 (Haber et al., 1989) describes a urinary catheter in which the valve means that controls the flow of urine through the catheter includes an inflatable balloon for occluding the passage of urine. U.S. Pat. No. 5,522,801 (Wang, 1996) provides a urethral catheter in which the outside surface of the drainage lumen comprises a longitudinal groove that, when covered with a sheath, acts as an inflation and deflation lumen for a balloon.

U.S. Pat. No. 6,254,570 (Rutner et al., 2001) discloses a drainage catheter with a balloon disposed near the distal end of the tubular member proximal to the external drainage port, which is on the distal end of the tubular member. U.S. Pat. No. 7,967,811 (Kumar, 2011) describes a Foley-type catheter in which the distal end of the elongated tube has a membrane that can be inflated into a balloon that retains the distal end in the interior of a bladder. U.S. Pat. No. 8,241,247 (Meek et al., 2012) and U.S. Pat. No. 8,641,665 (Meek et al., 2014) involves a drainage catheter having a drainage lumen, an inflation lumen that conveys inflating fluid from a proximal fluid supply element to a distal fluid acceptor balloon, and a sleeve that is secured about the proximal fluid supply element and the proximal fluid drain coupling to provide a fluid impervious coating.

U.S. Pat. No. 8,409,166 (Wiener et al., 2013) discloses an access device (or catheter) comprised of a cannula with a wall-piercing trocar within the lumen and two axially spaced inflatable balloons that engage the wall, thereby securing the cannula and sealing the puncture site. In one application, the invention is used as a biliary tree access cannula in which the two balloons rest against the internal and external walls of the gallbladder. U.S. Pat. No. 8,500,684 (Gardner et al., 2013) and U.S. Pat. No. 9,227,042 (Gardner et al., 2016) both involve a balloon catheter with a fluid drainage lumen, a balloon inflation lumen, a balloon portion disposed about the distal end of the catheter shaft, and a release device comprising an activating member that is attached to a tether to enable fluid flow from the balloon portion into the fluid drainage lumen.

U.S. Pat. No. 8,591,497 (Pinchuk et al., 2013) and U.S. Pat. No. 9,005,165 (Kaiser et al., 2015) disclose a safety catheter with a flexible, multi-lumen shaft that defines a drain lumen, a distal hollow balloon portion, at least one inflation lumen, and a drainage port. A hollow stretch valve is coaxially disposed in the inflation lumen, the drainage lumen, or both to prevent fluid from passing through the drainage port when the valve is in a steady (non-stretched) state. When the proximal end of the catheter is stretched, a sliding portion of the stretch valve slides within the valve to permit fluid to pass through the balloon drainage port and/or wall of the catheter. U.S. Patent Application Pub. No. 2004/0044307 (Richardson et al.) describes a urinary catheter with an outer layer having an inflatable portion for forming a locating balloon and an inner layer with having another inflatable portion that acts as an internal valve.

### BRIEF SUMMARY OF THE INVENTION

The present invention is a catheter comprising: an elongated tube having a central lumen, a proximal end, and a distal end terminating in a tip; a balloon and a balloon lumen that is in fluid communication with the balloon, the balloon being situated at a point along the elongated tube between the proximal and distal ends of the elongated tube; and a catheter hub that is connected to the proximal end of the elongated tube, the catheter hub being in fluid communication with the central lumen and the balloon lumen; wherein the elongated tube comprises an end hole at the tip of the distal end of the elongated tube; wherein the elongated tube comprises one or more side holes between the balloon and the hub; and wherein there is no fluid communication between the central lumen and the balloon lumen. In an alternate embodiment, the present invention is a catheter comprising: an elongated tube having a central lumen, a proximal end, and a distal end terminating in a tip; a balloon and a balloon lumen that is in fluid communication with the balloon, the balloon being situated at a point along the elongated tube between the proximal and distal ends of the elongated tube; and a catheter hub that is connected to the proximal end of the elongated tube, the catheter hub being in fluid communication with the central lumen and the balloon lumen; wherein the elongated tube comprises one or more side holes between the balloon and the tip of the distal end of the elongated tube; wherein the elongated tube comprises one or more side holes between the balloon and the hub; and wherein there is no fluid communication between the central lumen and the balloon lumen.

In another preferred embodiment, the present invention is a catheter comprising: an elongated tube having a central lumen, a proximal end, and a distal end terminating in a tip; a balloon and a balloon lumen that is in fluid communication with the balloon, the balloon being situated at a point along the elongated tube between the proximal and distal ends of the elongated tube; and a catheter hub that is connected to the proximal end of the elongated tube, the catheter hub being in fluid communication with the central lumen and the balloon lumen; wherein the elongated tube comprises a plurality of side holes between the balloon and the tip of the distal end of the elongated tube; wherein the elongated tube comprises a plurality of side holes between the balloon and the hub; and wherein there is no fluid communication between the central lumen and the balloon lumen.

In one embodiment, the plurality of side holes between the balloon and the tip of the distal end of the elongated tube are arranged in a linear pattern along a single cardinal plane of the elongated tube. In another embodiment, the plurality of side holes between the balloon and the tip of the distal end of the elongated tube are arranged in a spiral pattern along two cardinal planes of the elongated tube, the two cardinal planes being separated from one another by one hundred eighty degrees. In another embodiment, the plurality of side holes between the balloon and the tip of the distal end of the elongated tube are arranged in a spiral pattern along three cardinal planes of the elongated tube, the three cardinal planes being separated from one another by one hundred twenty degrees. In another embodiment, the plurality of side holes between the balloon and the tip of the distal end of the elongated tube are arranged in a spiral pattern along four cardinal planes of the elongated tube, the four cardinal planes being separated from one another by ninety degrees. In another embodiment, the plurality of side holes between the balloon and the tip of the distal end of the elongated tube are arranged in a spiral pattern along five cardinal planes of the elongated tube, the five cardinal planes being separated from one another by seventy-two degrees. In another embodiment, the plurality of side holes between the balloon and the tip of the distal end of the elongated tube are arranged in a spiral pattern along six cardinal planes of the elongated tube, the six cardinal planes being separated from one another by sixty degrees.

In one embodiment, the plurality of side holes between the balloon and the hub are arranged in a linear pattern along a single cardinal plane of the elongated tube. In another embodiment, the plurality of side holes between the balloon and the hub are arranged in a spiral pattern along two cardinal planes of the elongated tube, the two cardinal planes being separated from one another by one hundred eighty degrees. In another embodiment, the plurality of side holes between the balloon and the hub are arranged in a spiral pattern along three cardinal planes of the elongated tube, the three cardinal planes being separated from one another by one hundred twenty degrees. In another embodiment, the plurality of side holes between the balloon and the hub are arranged in a spiral pattern along four cardinal planes of the elongated tube, the four cardinal planes being separated from one another by ninety degrees. In another embodiment, the plurality of side holes between the balloon and the hub are arranged in a spiral pattern along five cardinal planes of the elongated tube, the five cardinal planes being separated from one another by seventy-two degrees. In another embodiment, the plurality of side holes between the balloon and the hub are arranged in a spiral pattern along six cardinal planes of the elongated tube, the six cardinal planes being separated from one another by sixty degrees.

In a preferred embodiment, the one or more side holes are in the range of 0.1 to 15 millimeters in diameter.

### BRIEF DESCRIPTION OF THE DRAWINGS

FIG. 1 is an illustration of the present invention in use as a biliary catheter.

FIG. 2 is an illustration of the present invention in use as a ureteral catheter.

FIG. 3 is a perspective view of the biliary catheter embodiment of the present invention.

FIG. 4 is a perspective view of the ureteral catheter embodiment of the present invention.

FIG. 5 is a partial perspective view of a section of the elongated tube of the present invention showing a first configuration of the primary and balloon lumens and a first configuration of the side holes.

FIG. 6 is a section view of the first configuration of the primary and balloon lumens of the present invention and the first configuration of the side holes shown in FIG. 5.

FIG. 7 is a section view of a second configuration of the primary and balloon lumens of the present invention shown without side holes.

FIG. 8 is a section view of a third configuration of the primary and balloon lumens of the present invention shown without side holes.

FIG. 9 is a section view of a fourth configuration of the primary and balloon lumens of the present invention shown without side holes.

FIG. 10A is a partial perspective view of a section of the elongated tube of the present invention showing the first configuration of the primary and balloon lumens with a second configuration of the side holes.

FIG. 10B is a section view of the first configuration of the primary and balloon lumens of the present invention and the second configuration of the side holes shown in FIG. 10A.

FIG. 11A is a partial perspective view of a section of the elongated tube of the present invention showing the first configuration of the primary and balloon lumens with a third configuration of the side holes.

FIG. 11B is a section view of the first configuration of the primary and balloon lumens of the present invention and the third configuration of the side holes shown in FIG. 11A.

FIG. 12A is a partial perspective view of a section of the elongated tube of the present invention showing the first configuration of the primary and balloon lumens with a fourth configuration of the side holes.

FIG. 12B is a section view of the first configuration of the primary and balloon lumens of the present invention and the fourth configuration of the side holes shown in FIG. 12A.

FIG. 13A is a partial perspective view of a section of the elongated tube of the present invention showing the first configuration of the primary and balloon lumens with a fifth configuration of the side holes.

FIG. 13B is a section view of the first configuration of the primary and balloon lumens of the present invention and the fifth configuration of the side holes shown in FIG. 13A.

FIG. 14A is a partial perspective view of a section of the elongated tube of the present invention showing the first configuration of the primary and balloon lumens with a sixth configuration of the side holes.

FIG. 14B is a section view of the first configuration of the primary and balloon lumens of the present invention and the sixth configuration of the side holes shown in FIG. 14A.

### REFERENCE NUMBERS

1 Catheter hub (proximal)
2 Catheter tip (distal)
3 Balloon
4 Elongated tube
5 Side holes
6 End hole
7 Central or primary lumen
8 Balloon lumen

### DETAILED DESCRIPTION OF INVENTION

### A. Overview

The present invention is a drainage catheter with a balloon and balloon lumen, at least one drainage hole (a side hole) along the catheter length between the catheter hub (or proximal end) and the balloon and at least one drainage hole (either a side hole or an end hole) along the catheter length between the balloon and the catheter tip (or distal end). Fluid and material that enters the main catheter lumen via a hole is able to pass through the catheter across the segment where the balloon is mounted to the opposite side of the catheter and out a hole on the distal end. This allows the catheter to function as a traditional drainage catheter, while simultaneously the balloon serves the additional function of providing a method for balloon dilation of a stricture.

Traditional balloon catheters, some of which are known as angioplasty balloon catheters, are tubular structures with a central lumen running from a hub on one end to an end hole on the other end of the catheter. Through this central lumen, a wire can be passed (for positioning and navigation of the balloon catheter) or fluids can be injected. These catheters serve the sole purpose of balloon dilation across a narrowed segment of vessel or other tubular structure. This balloon dilation is achieved by a second lumen, which extends along or through the catheter and connects the balloon mounted on the catheter shaft with a second hub (or port) on the proximal end of the catheter. The balloon is dilated up to a certain diameter as fluid is instilled in the balloon via the inflation lumen. Existing balloon catheters are designed for the sole purpose of dilation and not drainage; that is, they lack the necessary side holes along their length for fluids to enter the catheter along its length. The only way for fluids to flow in the main lumen of the catheter is from the hub to a side or end hole on the distal side of the balloon. As such, existing catheters cannot provide any drainage or fluid entry along the catheter course on the proximal side of the balloon.

Drainage catheters with balloons do exist, but they do not provide proximal-side drainage as in the present invention. One example of such a catheter is the Foley bladder catheter, in which the balloon is designed to act as a retaining device, not a dilation device. This type of catheter has a tip beyond the level of the balloon (on the distal side of the balloon) with one or more holes that allow fluids to pass into the tube, drain past the balloon, and retrograde to the hub of the catheter. There are no side holes along the tube between the hub and the balloon to allow fluids to enter the catheter on the section of catheter between hub and the balloon, pass through the catheter to the area beyond the balloon, and exit on the distal end of the catheter. Current drainage catheters with retaining balloons can serve the function of draining a cavity (such as the bladder) to the outside of the body; however, they cannot drain a pathway or structures within the body along the course of the catheter to the distal tip of the catheter such as the bile tree or the urinary tract. Additionally, the balloons on these drainage catheters are not designed dilate a tract; rather, they are larger than the pathway through which the catheter passes and, once inflated, retain the catheter from being inadvertently pulled backward.

Percutaneous feeding tubes, also known as g-tubes (gastrostomy tubes or jejunostomy tubes), are similar to Foley catheters. These tubes are used for feeding purposes and/or to decompress the stomach. They are placed from the skin surface into the stomach through a surgically created tract. These catheters are similar in design to Foley catheters in that there is a balloon on the distal tip of the catheter that retains the catheter in the body cavity (stomach or bowel) but does not dilate the tract. As with the Foley catheter, there are holes on the segment of tube distal to the balloon and another hole at the hub of the catheter for flushing or instillation of food into the target organ. There are no holes along the side of the catheter between the balloon and the hub, which would allow drainage of a structure along the path of the catheter.

In contrast to the balloon catheters described above, traditional drainage catheters have side holes along their length and are designed allow entry of fluid into the central lumen from the structure through which they are traversing or passing; the fluid exits out the distal length of catheter into the targeted terminus. An example of this type of catheter is the biliary drainage catheter, in which the catheter side holes allow bile in the bile ducts in the liver to enter the main lumen of the catheter, pass through the lumen, and exit the distal segment of the catheter either through side holes or the end hole on the segment, which terminates in the small bowel. These catheters also have a hub on the proximal end (similar to an angioplasty catheter) that allows them to be advanced and positioned over a wire or that enables fluids to be instilled into the main lumen of the catheter for the purpose of flushing the catheter. Additionally, any fluids that enter the drainage catheter through holes along its length can also drain retrograde (defined as back toward the hub) and out of the hub to a syringe or bag in the event the physician wishes to drain externally or if the tube becomes clogged and needs pressure relieved until the clog can be fixed.

These traditional drainage catheters are used in the biliary tree, kidneys and ureters (the passageway draining the urine collection system of the kidney to the bladder). Drainage catheters are also used to drain abscesses and other cavities to the outside of the body. None of the currently available side hole drainage catheters has a balloon mounted on it for the purpose of both drainage and dilation. In fact, no currently available drainage catheters have balloons mounted on them in which there is at least one side hole along the catheter length between the catheter hub and the balloon and another hole (either a side hole or an end hole) along the catheter length between the balloon and the catheter tip.

As used herein, the term “balloon” means a reservoir that can be expanded by the instillation of fluid or gas into the reservoir. The balloon of the present invention may be of any configuration that suits the specific anatomy and application. The balloon may be of any shape, including, but not limited to, cylindrical, dumbbell-shaped, spherical, prism, cuboid, cube, pyramid or cone, any of which may have blunt, tapering or expanding shoulders/edges. These balloons may be distensible in either a binary fashion, either completely distended or non-distended, or distensible in an incremental fashion (any number of varying degrees of inflation). The balloon may also have multiple adjacent or concentric lumens such that variable configurations or degrees of inflation may be attained in the single region of mounted balloon. The balloon may be designed so that it is permanently, semi-permanently or intermittently distensible. The balloon may also be designed so that it is of any degree of compliance (compliance being the property of a material undergoing elastic deformation or change in volume when subjected to an applied force). The balloon profile may be in any configuration relative to the catheter, such as circumferentially relative to the outside of the catheter or eccentric to the outside of the catheter. The balloon material itself may be mounted/associated either circumferentially or eccentrically to the wall of the catheter. The balloon may have a separate wall or walls free from the catheter, a separate wall or walls joined to the catheter by some method of attachment, or a shared wall or walls with the catheter.

As used herein, the term “side hole” means a hole in the wall of the elongated tube that provides a fluid conduit between the space outside of the catheter and the primary lumen. As used herein, the term “end hole” means a hole at the distal tip of the elongated tube of the catheter that is in fluid communication with the primary lumen. The present invention is discussed in greater detail below with reference to the figures.

### B. Detailed Description of the Figures

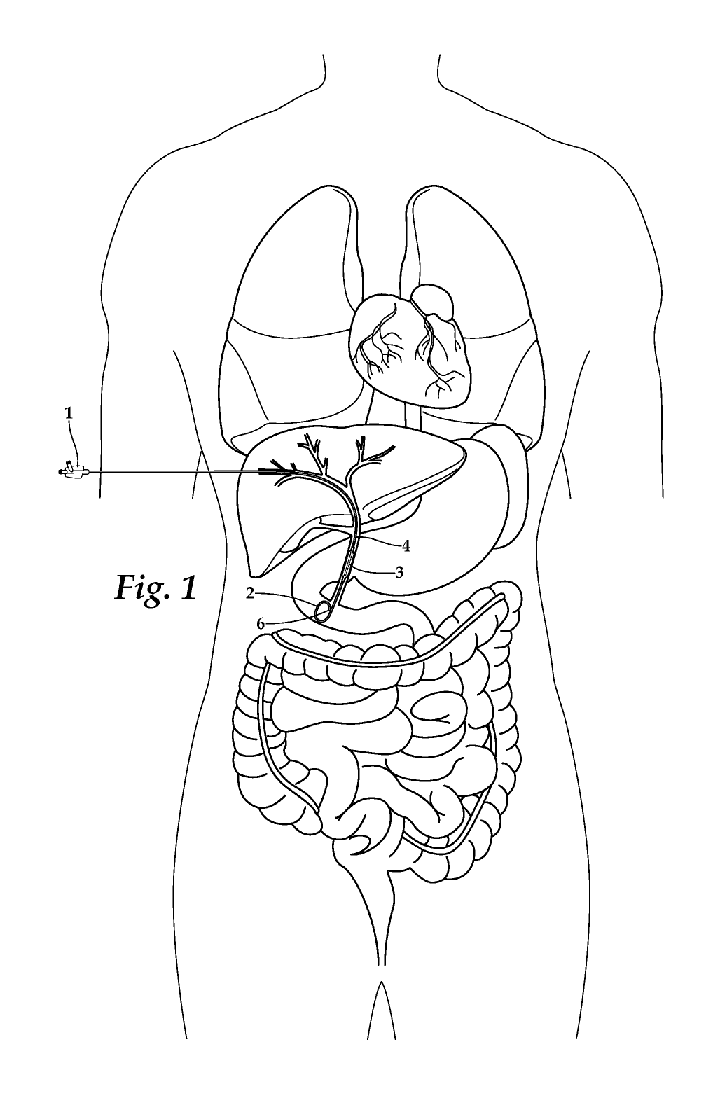

FIG. 1 is an illustration of the present invention in use as a biliary catheter. As shown in this figure, the catheter hub 1 is situated outside of the patient's body, and the catheter tip 2 resides in the small bowel. The balloon 3 is ideally situated so that it can dilate a stricture in the biliary tree.

FIG. 2 is an illustration of the present invention in use as a ureteral catheter. As shown in this figure, the catheter hub 1 is located outside of the patient's body, the catheter tip 2 is positioned within the bladder, and the balloon 3 is situated in the ureter at the point of stricture.

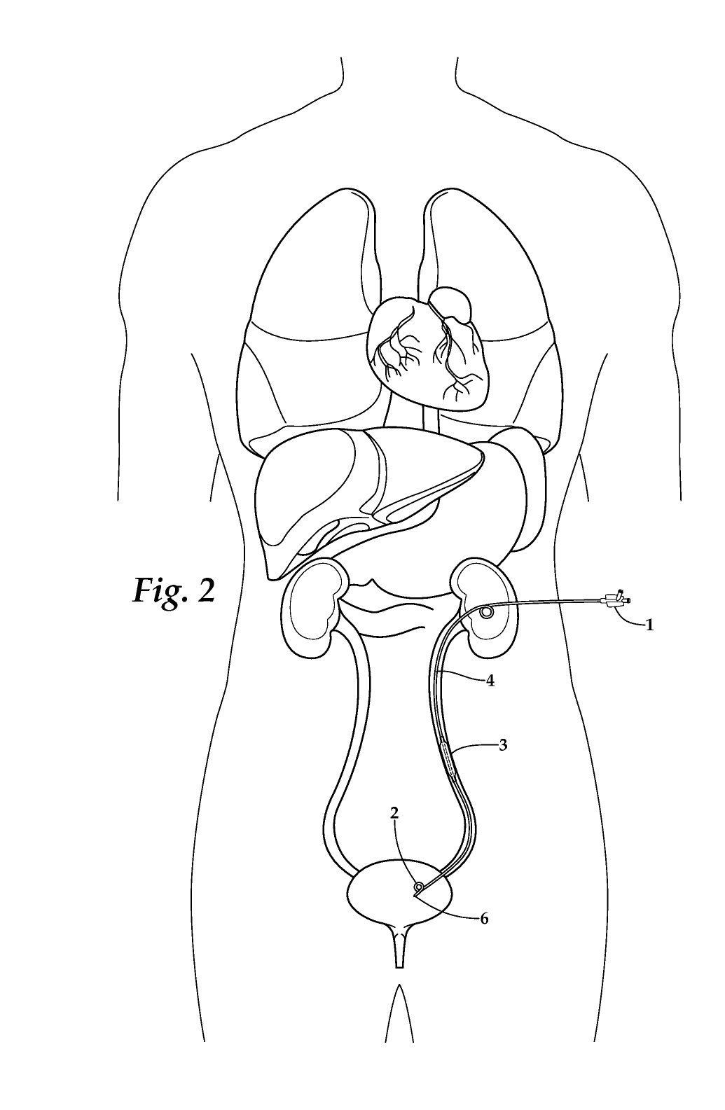

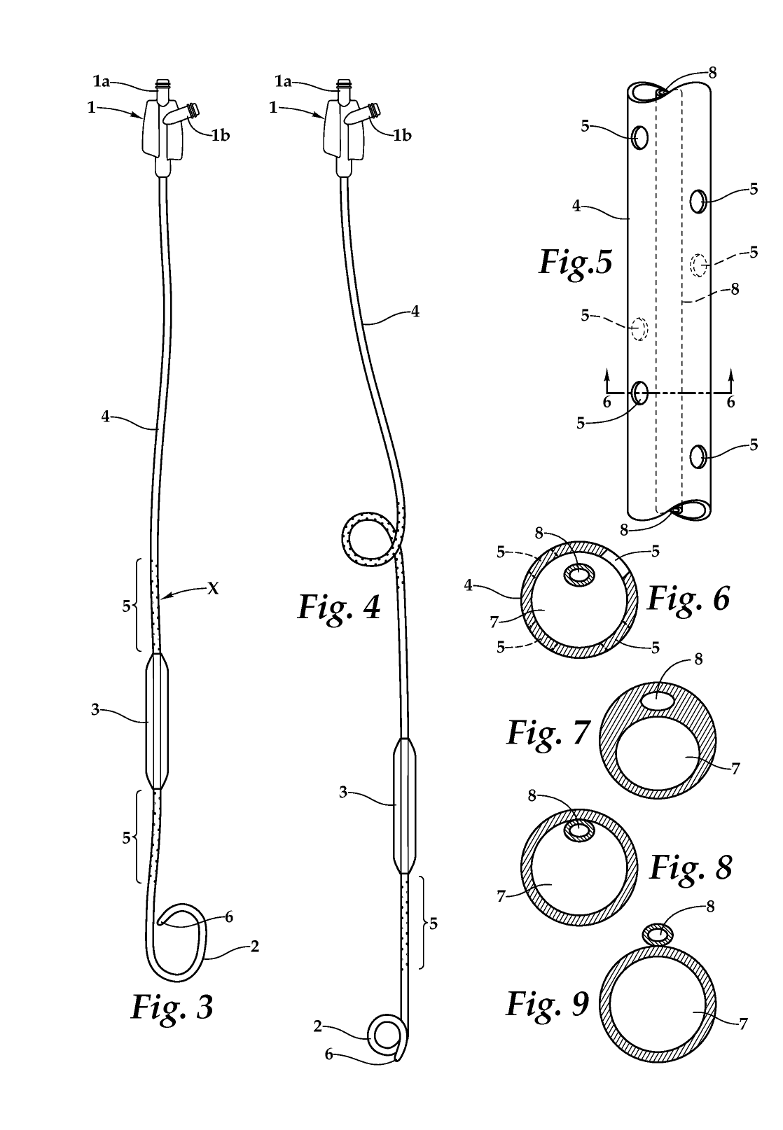

FIG. 3 is a perspective view of the biliary catheter embodiment of the present invention. The present invention is a catheter comprising a catheter hub 1, an elongated tube 4 with a tip 2 on the distal end of the tube, and a balloon 3 situated at any point along the elongated tube 4 between the catheter hub 1 and the tip 2. The present invention is not limited to any particular size or configuration of the hub 1 as long as it provides fluid communication to both the central lumen and the balloon lumen (see FIGS. 6-9). The hub 1 may be a single molded part, as shown in this figure, or it may be comprised of two separately molded parts (one of which is in fluid communication with the central lumen and the other of which is in fluid communication with the balloon lumen). As used herein, the term “hub” means any part or parts (in other words, it may refer to a single part or more than one part) to which a syringe or other delivery mechanism may be attached for the purpose of instilling the central and balloon lumens with fluid(s). In FIGS. 3 and 4, the first port 1a of the hub 1 is in fluid communication with the primary lumen 7, and the second port 1b of the hub 1 is in fluid communication with the balloon 3. Distension of the balloon 3 is effectuated by the instillation of fluid (including, but not limited to, liquids and gases) through the second port 1b from a temporarily or permanently engaged container. The first and/or second port 1a, 1b may comprise a valve, switch, stopcock or clamp to permit temporary or permanent closure of the central and/or balloon lumens.

The present invention is not limited to any particular shape, size, material or compliance (rigidity) of the balloon 3, as long as it is in fluid communication with the second port 1b of the hub 1. The balloon 3 is preferably sized so that it can be used to dilate the tract in which the catheter is placed. (as opposed to balloons that are used solely for retention purposes, in which case the balloons may be significantly larger than they are in the present invention).

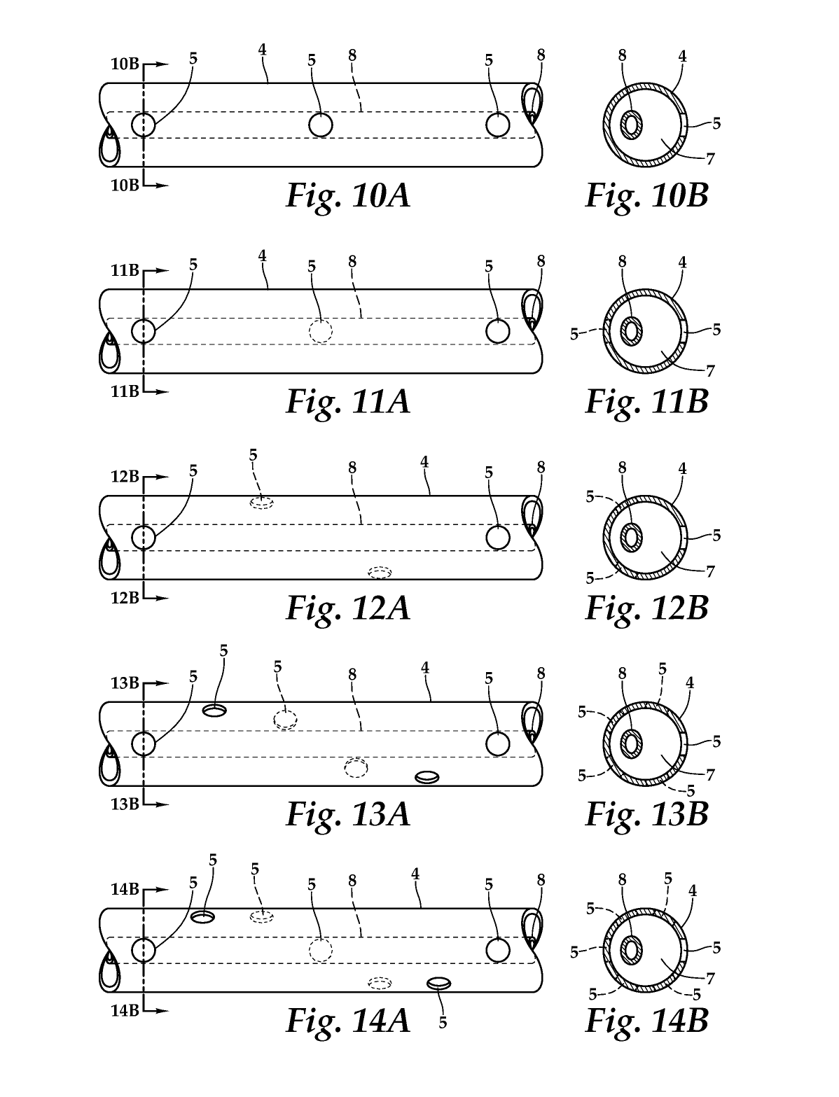

FIG. 4 is a perspective view of the ureteral catheter embodiment of the present invention. This embodiment is also comprised of a hub 1, elongated tube 4, balloon 3 and catheter tip 2, as described above. Both FIGS. 3 and 4 also show a first plurality of side holes 5 in the elongated tube 4 on the distal side of the balloon 3 (between the balloon and the tip) and a second plurality of side holes 5 in the elongated tube 4 on the proximal side of the balloon 3 (between the balloon and the hub). The present invention is not limited to any particular number or configuration of side holes, and several different embodiments are shown in FIGS. 10-15 below. The present invention does require, however, at least one hole (a side hole or an end hole) on the distal side of the balloon and at least one hole (a side hole) on the proximal side of the balloon. The end hole 6 is located on the distal end of the catheter tip 2. Both the end hole 6 and the side holes 5 allow fluid to enter or exit the central lumen 7 (see FIGS. 6-9) of the elongated tube 4. Although the balloon 3 is situated on the elongated tube 4, there are no side holes 5 on that portion of the elongated tube 4 along which the balloon 3 is positioned. The catheter tip 2 may be of any configuration, including, but not limited to, a locking loop, a non-locking loop, suture-secured, straight, pigtail, tapered, flared or blunt.

FIG. 5 is a partial perspective view of a section of the elongated tube of the present invention. This figure shows one particular configuration of the side holes; alternate configurations are shown in FIGS. 10A-14B. In the configuration shown in FIG. 5, the side holes 5 are arranged in a spiral pattern along four cardinal planes of the elongated tube 4 (the four planes being separated from one another by ninety degrees), at 0 degrees, 90 degrees, 180 degrees and 270 degrees, respectively. In a preferred embodiment, the side holes range in diameter from 0.1 mm to 15 mm. Although the holes are shown as circular, they may be of any geometry, including, but not limited to, circular, ovoid, rectangular, square, multi-lobed, teardrop or rhomboid.

FIG. 5 also shows the section line for FIG. 6. The section views shown in FIGS. 7, 8 and 9 are taken from the same position along the elongated tube 4 but show different configurations of the primary and balloon lumens; these figures are discussed more fully below. Note that the side holes have been omitted from FIGS. 7, 8 and 9 for illustration purposes only. Although all of the figures depict a single balloon lumen 8, the present invention could be implemented with more than one balloon 3 and more than one balloon lumen 8.

FIG. 6 is a section view of a first configuration of the primary and balloon lumens of the present invention. The present invention comprises both a central or primary lumen 7 that extends from the hub 1 to the tip 2 and a balloon or inflation lumen 8 that is in fluid communication with the balloon 3. The balloon lumen 8 extends from the hub 1 (which, as stated above, may be the same hub from which the primary lumen extends or an entirely separate hub) to the balloon 3; it does not extend distally past the balloon. For this reason, the portion or elongated tube 4 shown in FIG. 5 is taken from the proximal side of the balloon (designated as “X” on FIG. 3). In the present invention, the central lumen 7 is not in fluid communication with the balloon lumen 8; that is, the two lumens are completely separate along their entire length, and there is no fluid communication (or conduit) between the two. The primary lumen 7 is the void along the inside of the elongated tube 4 beginning at the hub 1 and terminating at either the most distal side hole or an end hole.

In FIG. 6, the balloon lumen 8 is inside of the central lumen 7 but free-floating within it. In FIG. 7, the balloon lumen 8 is integrated with or formed by the material of the wall of the primary lumen 7. The catheter wall itself may be made of a single material or any combination of materials. In FIG. 8, the balloon lumen 8 is inside of the central lumen 7 and affixed to the inside of the wall of the central lumen. In FIG. 9, the balloon lumen 8 runs along the outside of the wall of the central lumen 7. The present invention is not limited to any particular configuration of central lumen and balloon lumen as long as there is a central lumen that acts as a conduit of fluid from the hub 1 to the tip 2 (or to the distal-most side hole) and a balloon lumen that acts as a conduit of fluid from the hub to the balloon 3. As stated above, the two lumens are not in fluid communication with one another at any point.

FIG. 10A is a partial perspective view of a section of the elongated tube of the present invention showing the first configuration of the primary and balloon lumens with a second configuration of the side holes, and FIG. 10B is a section view of the first configuration of the primary and balloon lumens of the present invention and the second configuration of the side holes shown in FIG. 10A. In this embodiment, the side holes are arranged in a linear pattern along a single cardinal plane of the elongated tube 4.

FIG. 11A is a partial perspective view of a section of the elongated tube of the present invention showing the first configuration of the primary and balloon lumens with a third configuration of the side holes, and FIG. 11B is a section view of the first configuration of the primary and balloon lumens of the present invention and the third configuration of the side holes shown in FIG. 11A. In this embodiment, the side holes 5 are arranged in a spiral pattern along two cardinal planes of the elongated tube (the two planes being separated from one another by one hundred eighty degrees), at 0 degrees and 180 degrees, respectively.

FIG. 12A is a partial perspective view of a section of the elongated tube of the present invention showing the first configuration of the primary and balloon lumens with a fourth configuration of the side holes, and FIG. 12B is a section view of the first configuration of the primary and balloon lumens of the present invention and the fourth configuration of the side holes shown in FIG. 12A. In this embodiment, the side holes 5 are arranged in a spiral pattern along three cardinal planes of the elongated tube 4 (the three planes being separated from one another by one hundred twenty degrees), at 0 degrees, 120 degrees and 240 degrees, respectively.

FIG. 13A is a partial perspective view of a section of the elongated tube of the present invention showing the first configuration of the primary and balloon lumens with a fifth configuration of the side holes, and FIG. 13B is a section view of the first configuration of the primary and balloon lumens of the present invention and the fifth configuration of the side holes shown in FIG. 13A. In this embodiment, the side holes 5 are arranged in a spiral pattern along five cardinal planes of the elongated tube 4 (the five planes being separated from one another by seventy-two degrees), at 0 degrees, 72 degrees, 144 degrees, 216 degrees and 288 degrees, respectively.

FIG. 14A is a partial perspective view of a section of the elongated tube of the present invention showing the first configuration of the primary and balloon lumens with a sixth configuration of the side holes, and FIG. 14B is a section view of the first configuration of the primary and balloon lumens of the present invention and the sixth configuration of the side holes shown in FIG. 14A. In this embodiment, the side holes 5 are arranged in a spiral pattern along six cardinal planes of the elongated tube 4 (the six planes being separated from one another by sixty degrees), at 0 degrees, 60 degrees, 120 degrees, 180 degrees, 240 degrees and 300 degrees, respectively.

With the present invention, there are several possible pathways for fluids: (1) material can enter the catheter hub and pass through the primary lumen and exit out a hole (side hole and/or end hole) in the segment of the catheter that is distal to the balloon; (2) material can enter the catheter hub and pass through the primary lumen and exit out a side hole in the segment of the catheter that is proximal to the balloon; (3) material can enter the primary lumen through a side hole in the segment of the catheter that is proximal to the balloon and move retrograde toward and out of the catheter hub; (4) material can enter the primary lumen through a side hole in the segment of the catheter that is proximal to the balloon and move antegrade toward and out of a hole (side hole and/or end hole) in the segment of the catheter that is distal to the balloon; (5) material can enter the primary lumen through a hole (side hole and/or end hole) in the segment of the catheter that is distal to the balloon and move retrograde toward and out of a side hole in the segment of the catheter that is proximal to the balloon; and (6) material can enter the primary lumen through a hole (side hole and/or end hole) in the segment of the catheter that is distal to the balloon and move retrograde toward and out of the catheter hub. All of these pathways can occur while the balloon is in a deflated/non-distended or an inflated/distended state, and all of these pathways except (2) and (3) allow passage of material through the primary lumen across the segment of the catheter upon which the balloon is mounted. More than one of the foregoing pathways may occur simultaneously.

Although the preferred embodiment of the present invention has been shown and described, it will be apparent to those skilled in the art that many changes and modifications may be made without departing from the invention in its broader aspects. The appended claims are therefore intended to cover all such changes and modifications as fall within the true spirit and scope of the invention.

I claim:

1. A catheter comprising:
(a) an elongated tube having a central lumen, a proximal end, and a distal end terminating in a tip;
(b) a balloon and a balloon lumen that is in fluid communication with the balloon, the balloon being situated at a point along the elongated tube between the proximal and distal ends of the elongated tube; and
(c) a catheter hub that is connected to the proximal end of the elongated tube, the catheter hub being in fluid communication with the central lumen and the balloon lumen;
wherein the elongated tube comprises an end hole at the tip of the distal end of the elongated tube;
wherein the elongated tube comprises one or more side holes between the balloon and the hub; and
wherein there is no fluid communication between the central lumen and the balloon lumen.

2. A catheter comprising:
(a) an elongated tube having a central lumen, a proximal end, and a distal end terminating in a tip;
(b) a balloon and a balloon lumen that is in fluid communication with the balloon, the balloon being situated at a point along the elongated tube between the proximal and distal ends of the elongated tube; and
(c) a catheter hub that is connected to the proximal end of the elongated tube, the catheter hub being in fluid communication with the central lumen and the balloon lumen;
wherein the elongated tube comprises one or more side holes between the balloon and the tip of the distal end of the elongated tube;
wherein the elongated tube comprises one or more side holes between the balloon and the hub; and
wherein there is no fluid communication between the central lumen and the balloon lumen.

3. A catheter comprising:
(a) an elongated tube having a central lumen, a proximal end, and a distal end terminating in a tip;
(b) a balloon and a balloon lumen that is in fluid communication with the balloon, the balloon being situated at a point along the elongated tube between the proximal and distal ends of the elongated tube; and
(c) a catheter hub that is connected to the proximal end of the elongated tube, the catheter hub being in fluid communication with the central lumen and the balloon lumen;
wherein the elongated tube comprises a plurality of side holes between the balloon and the tip of the distal end of the elongated tube;
wherein the elongated tube comprises a plurality of side holes between the balloon and the hub; and
wherein there is no fluid communication between the central lumen and the balloon lumen.

4. The catheter of claim 3, wherein the plurality of side holes between the balloon and the tip of the distal end of the elongated tube are arranged in a linear pattern along a single cardinal plane of the elongated tube.

5. The catheter of claim 3, wherein the plurality of side holes between the balloon and the tip of the distal end of the elongated tube are arranged in a spiral pattern along two cardinal planes of the elongated tube, the two cardinal planes being separated from one another by one hundred eighty degrees.

6. The catheter of claim 3, wherein the plurality of side holes between the balloon and the tip of the distal end of the elongated tube are arranged in a spiral pattern along three cardinal planes of the elongated tube, the three cardinal planes being separated from one another by one hundred twenty degrees.

7. The catheter of claim 3, wherein the plurality of side holes between the balloon and the tip of the distal end of the elongated tube are arranged in a spiral pattern along four cardinal planes of the elongated tube, the four cardinal planes being separated from one another by ninety degrees.

8. The catheter of claim 3, wherein the plurality of side holes between the balloon and the tip of the distal end of the elongated tube are arranged in a spiral pattern along five cardinal planes of the elongated tube, the five cardinal planes being separated from one another by seventy-two degrees.

9. The catheter of claim 3, wherein the plurality of side holes between the balloon and the tip of the distal end of the elongated tube are arranged in a spiral pattern along six cardinal planes of the elongated tube, the six cardinal planes being separated from one another by sixty degrees.

10. The catheter of claim 3, wherein the plurality of side holes between the balloon and the hub are arranged in a linear pattern along a single cardinal plane of the elongated tube.

11. The catheter of claim 3, wherein the plurality of side holes between the balloon and the hub are arranged in a spiral pattern along two cardinal planes of the elongated tube, the two cardinal planes being separated from one another by one hundred eighty degrees.

12. The catheter of claim 3, wherein the plurality of side holes between the balloon and the hub are arranged in a spiral pattern along three cardinal planes of the elongated tube, the three cardinal planes being separated from one another by one hundred twenty degrees.

13. The catheter of claim 3, wherein the plurality of side holes between the balloon and the hub are arranged in a spiral pattern along four cardinal planes of the elongated tube, the four cardinal planes being separated from one another by ninety degrees.

14. The catheter of claim 3, wherein the plurality of side holes between the balloon and the hub are arranged in a spiral pattern along five cardinal planes of the elongated tube, the five cardinal planes being separated from one another by seventy-two degrees.

15. The catheter of claim 3, wherein the plurality of side holes between the balloon and the hub are arranged in a spiral pattern along six cardinal planes of the elongated tube, the six cardinal planes being separated from one another by sixty degrees.

16. The catheter of claim 1, 2 or 3, wherein the one or more side holes are in the range of 0.1 to 15 millimeters in diameter.
